(19) United States
(12) Patent Application Publication
(10) Pub. No.: US 2020/0392059 A1
(43) Pub. Date: Dec. 17, 2020

(54) Mobile Crop Processing System and Method

(71) Applicant: Ambient LLC, Boise, ID (US)

(72) Inventors: Wren Van Bockel, Boise, ID (US); Ryan Eldridge, Boise, ID (US)

(73) Assignee: Ambient LLC, Boise, ID (US)

(21) Appl. No.: 16/900,270

(22) Filed: Jun. 12, 2020

Related U.S. Application Data
(60) Provisional application No. 62/984,044, filed on Mar. 2, 2020.
(60) Provisional application No. 62/860,429, filed on Jun. 12, 2019.

(51) Int. Cl. C07C 37/72; B01D 1/22; B01F 15/02; B01D 21/26; B01D 11/02; B01D 21/28; C07C 37/68

(52) U.S. Cl. CPC C07C 37/72; B01D 1/22; B01F 15/0289; B01D 21/262; A01D 45/065; B01D 11/0257; B01D 21/283; C07C 37/685; B01D 11/0288

(57) ABSTRACT

A mobile crop processing system includes a self-propelled harvester that shears and chops plant material, a mobile extraction assembly that follows the harvester and receives the chopped plant material to produce a crude oil output while discarding unwanted moisture and biomass in the field, a field-based extraction assembly that separates the crude oil into reclaimed solvent and essential oil, and a transporter for carrying containers of crude oil and solvent between the mobile and field-based extraction assemblies. The mobile extraction assembly includes a pre-treatment assembly for mixing plant material with solvent, a dewatering and maceration assembly for removing moisture from the plant material, and a separation assembly for removing additional moisture and biomass. Using the system, crops can be harvested and processed in the field when they are ready to be cut. The system can operate continuously until a desired portion of the field has been cut and processed.

### CROSS-REFERENCE TO RELATED APPLICATIONS

This application claims the benefit of co-pending provisional U.S. Application No. 62/984,044 filed Mar. 2, 2020 and co-pending provisional U.S. Application No. 62/860,429 filed Jun. 12, 2019.

### FIELD OF THE INVENTION

The present invention relates to a novel and useful mobile crop processing system for removing moisture and extracting oils from plant materials such as hemp. The present invention also relates to a method of using the system and a method of farming plant materials such as industrial hemp using the system.

### BACKGROUND OF THE INVENTION

Cannabidiol including terpenes (CBD), one of many compounds found in the Cannabis sativa plant, is generally recognized for its health benefits. Typically, CBD is concentrated in CBD oil and does not give the psychotropic feeling generally associated with tetrahydrocannabinol (THC), the most active ingredient in the Cannabis sativa plan. CBD has been found to reduce chronic pain and inflammation and to reduce or prevent withdrawal symptoms when quitting smoking or other substances. It has also been shown effective for treating epilepsy, neurodegeneration, neuronal injury, and psychiatric diseases. Some researchers have further found that CBD suppresses the growth of some cancerous cells and promotes their destruction. Additionally, CBD may reduce anxiety-related behaviors in people with post-traumatic stress, general anxiety, panic, social anxiety, and obsessive-compulsive disorders. Other promising research suggests that CBD may be useful for treating Type 1 diabetes, acne, and Alzheimer's disease.

The CBD most commonly used for its health benefits is extracted from industrial hemp plants. In order to satisfy legal requirements, industrial hemp contains only a trace amount of THC (less than 0.3% THC) in addition to CBD. There are many varietals of industrial hemp including some specifically bred to be high-resin cannabis and therefore CBD-rich while maintaining only trace amounts of THC. CBD-rich hemp differs from hemp grown for seed or fiber by being bred for larger flower size and quantity of flowers, making its shape closer to a Christmas tree. There is no established method of harvesting CBD-rich hemp on a large scale, but common methods include being sickled at the base by hand or tractor and left to dry in the field or removed from the ground and brought to an extraction facility, dry the whole plant or strip flowers and dry the flowers only, then chopping it or milling it to a powder. At the facility, solvents such as ethanol or CO2 extraction is often used, which is among the cleanest ways to extract CBD oil from the hemp plant. CO2 extraction involves filtering plants through a series of chambers that control temperature and pressure to isolate CBD at a 90% efficiency. Alternatively, ethanol extraction involves introducing the solvent ethanol to the hemp to extract CBD, which produces a very high volume of full spectrum hemp extract without unwanted components. After extraction with ethanol or CO2, the oil undergoes chromatography to remove unwanted phytochemicals. Some CBD oils also undergo decarboxylation to increase bioavailability and winterization to remove fats. CBD oil can be consumed directly or it can be mixed with carrying oils such as hemp seed oil or coconut oil, turned into capsules, or processed into a powder or slab isolate form.

As the popularity of CBD products continues to rise, farmers are facing challenges in the field. The plants are commonly seeded or transplanted as seedlings at 5 foot intervals and then grown to 5′-6′ tall plants before harvesting. The plants also may be topped to increase leaf production, which causes them to be bushy and top-heavy. Additionally, some plants are dried in the field before harvesting. Unfortunately, as a result, the plants are particularly susceptible to wind, hail, and bird damage or rotting if excess precipitation occurs. One method to combat such damage is to swath the plants, but swathing introduces other hazards such as considerable risk of seed quality loss and increased levels of microbial contamination if hail or rain flattens the swath onto the ground. Alternatively, the plants can be harvested before drying to minimize environmental damage, but then they require additional aeration and drying before the leaves can be stored or stripped for processing. Most large scale farmers will use natural gas or propane dryers. Accordingly, space is needed to accommodate timely aeration and drying. Drying, for example, may require sufficient space where the stalks can be hung upside down, preferably in a cool and dark location with sufficient air movement. Accordingly, it would be desirable to minimize damage to hemp crops due to prolonged exposure to environmental hazards by providing a system and method allowing for earlier harvesting. It would also be desirable to minimize the need for additional space and time for drying hemp that was harvested with higher moisture content.

### SUMMARY OF THE INVENTION

In accordance with the present invention a novel and useful mobile crop processing system and method of farming plant materials such as industrial hemp using the system is provided. The mobile crop processing system includes a self-propelled forage harvester with a chopper head or a modified picker, depending on the crop, that shears and chops plant material such as hemp in the field and a mobile and field-based extraction system having a mobile extraction assembly that preferably includes a pre-treatment assembly, a field-based extraction assembly, and a transporter that moves products and materials between the mobile extraction assembly and field-based extraction assembly. The forage harvester preferably pulls one or more trailers, on which sits the mobile extraction assembly of components. Preferably the forage harvester pulls two trailers, one on which sits the pre-treatment assembly components of the mobile extraction assembly and another on which sits the additional mobile extraction components including an inlet hopper or an optional feed hopper, a dewatering and maceration assembly, and a first separation assembly. The plant material is delivered to the pre-treatment assembly in the field as it is being cut via a chute on the forage harvester. The output of the pre-treatment assembly is then delivered directly and preferably with a screw auger to the inlet or feed hopper, which holds the pre-treated plant material or slurry and delivers it to the mobile extraction assembly for immediate processing in the field. Optionally, the plant material can be delivered directly to the dewatering and maceration assembly where pre-treatment is not wanted. Also optionally, where field conditions might prevent excessive movement of the mobile extraction assembly components, they can be positioned stationary near or on the field and the forest harvester may pull a third trailer for collecting and moving crops. The third trailer can receive the plant material and then deliver it to the mobile extraction assembly for processing.

In addition to the pre-treatment assembly, the mobile extraction assembly includes a dewatering and maceration assembly, a mobile or first separation assembly, and a transport container. With the dewatering and maceration assembly, the mobile extraction assembly dewaters the pre-treated plant material (or untreated plant material where no pre-treatment assembly is present) through a series of screw presses. Approximately 4,000-8,000 lbs. of plant material can be dewatered per hour through the series of screw press operations, and if needed for better recoveries, it can be followed by additional maceration with a solvent. Next, with the first separation assembly, a first crude oil consisting of oil, fats, waxes, terpenes, solvent, and additional water is then separated from the resulting biomass with a separator such as a decanter centrifuge or ambient filtration skid after which a refined crude oil consisting of oil and solvent is separated from the first crude oil with an oil water separator and collected in a collection tank. The refined crude oil is transferred from the collection tank to the transport container for transport with a transport vehicle to the field-based extraction assembly.

The field-based extraction assembly includes an additional separator, a container for collecting essential oil, and a container for collecting reclaimed solvent. After the refined crude oil is delivered to the field-based extraction assembly, the cannabinoid/terpene-rich oil or essential oil is separated from the solvent using a wiped film evaporator. The essential oil is collected in the essential oil container for further processing and reclaimed solvent is collected in the reclaimed solvent container for reuse by the mobile extraction assembly in the solvent extraction process.

With both the mobile and field-based extraction assemblies, any unwanted moisture and biomass encountered during processing is discarded in the field or collected in a container or other receptacle for other purposes if desired. The process from chopper or modified picker to output of refined crude oil takes approximately 30-60 minutes. The process from refined crude oil to essential oil takes approximately 15-30 minutes.

A method of farming plant material such as industrial hemp using the mobile crop processing system of the present invention involves first planting and growing suitable hemp for harvesting. Once the hemp is ready to be cut, the mobile crop processing system is brought to the field. The hemp flower is harvested and then may be additionally cut into pieces or plant material using the chopper/modified picker and forage harvester of the mobile crop processing system and preferably delivered via the forage harvester's chute to the pre-treatment assembly of the mobile extraction assembly. After pre-treating the hemp, it is conveyed to the inlet or feed hopper of the mobile extraction assembly. The additional components of the mobile extraction assembly process the plant material and produce refined crude oil that is then transferred with the transporter to the field-based extraction assembly. The field-based extraction assembly processes the crude oil and produces essential oil and reclaimed solvent. With both the mobile and field-based extraction assemblies, any unwanted moisture and biomass is preferably discarded into the field. The mobile crop processing system operates as needed until the entire field of hemp, or until a desired portion of the field, has been cut and processed. After the field has been cleared, additional hemp can be planted and the cycle can be repeated. For a field of about 120 acres, the mobile crop processing system can process 4,000-8,000 lbs. of plant mass per hour for a total processing time of about 60-120 hours. Over one year of continuous farming, it is anticipated that over 20,000 lbs. of essential oil can be recovered at a yield rate of about 76%. While the method is described with respect to farming hemp, other plant materials can be farmed according to the same method.

### BRIEF DESCRIPTION OF THE SEVERAL VIEWS OF THE DRAWING

FIG. 1A is a side view of the preferred embodiment of the mobile crop processing system of the present invention.

FIG. 1B is a side view of an alternate embodiment of the mobile crop processing system of the present invention.

FIG. 2A is a schematic of the preferred embodiment of the mobile and field-based extraction assemblies according to a first embodiment of the present invention.

FIG. 2B is a schematic of the mobile and field-based extraction assemblies according to a second embodiment of the present invention.

FIG. 2C is a schematic of the mobile and field based extraction assemblies according to a third embodiment of the present invention.

FIG. 3A is a flow chart of the mobile and field-based extraction process according to the first embodiment of the present invention.

FIG. 3B is a schematic of the mobile and field-based extractor components according to the second embodiment of the present invention.

FIG. 4 is a flow chart of the method of using the mobile crop processing system to farm industrial hemp according to the present invention.

For a better understanding of the invention reference is made to the following detailed description of the preferred embodiments of the invention which should be taken in conjunction with the above described drawings.

### DETAILED DESCRIPTION OF THE INVENTION

Various aspects of the present invention will evolve from the following detailed description of the preferred embodiments thereof which should be referenced to the prior described drawings.

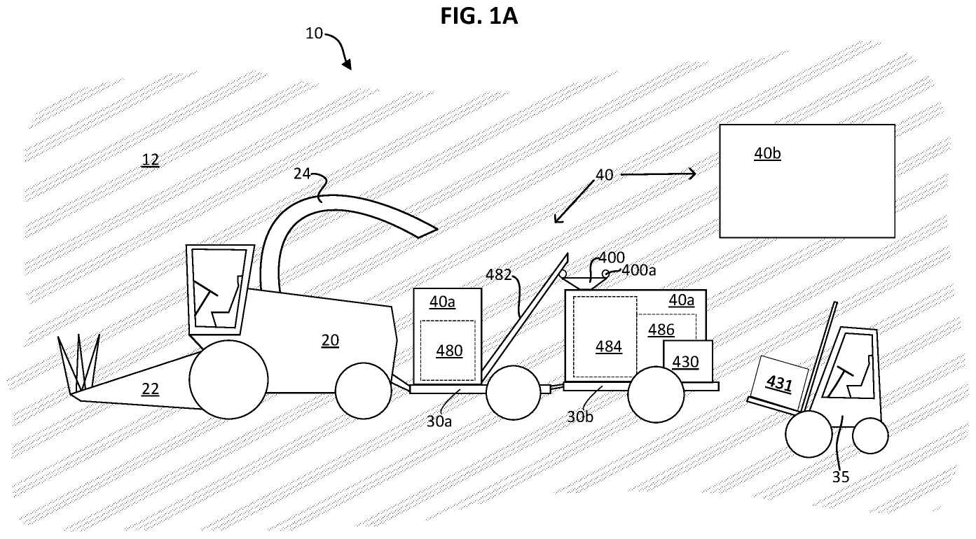

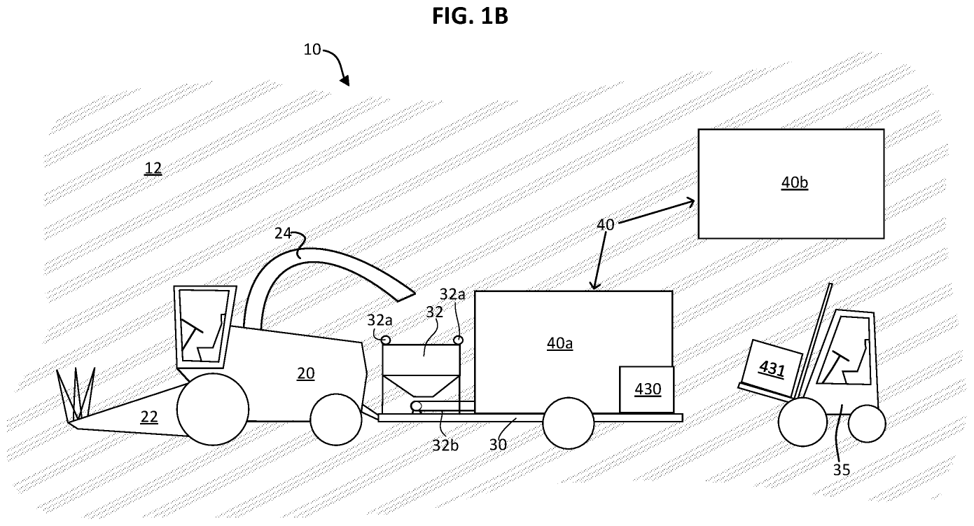

The mobile crop processing system 10 of the present invention is illustrated in FIGS. 1A-1B. As shown, the mobile crop processing system includes a forage harvester 20, a trailer 30, a transporter 35, optional movable containers 430 and 431, and a mobile and field-based extraction system 40 having a mobile extraction assembly 40a and a field-based extraction assembly 40b. The forage harvester 20 of the present invention is preferably self-propelled such that it can operate on a field 12 and includes a chopper head or modified picker 22 and a chute 24. Preferably, for the present invention, the forage harvester 20 and chopper head 22 are farm implements such as silage harvesters, foragers, and choppers that can harvest forage plants and chop into small pieces, and chopper head 22 shall be understood to include pickers, rakes, combines, threshers and other types of farm equipment capable of chopping, cutting, and harvesting. More preferably, forage harvester 20 and chopper 22 are capable of shearing and chopping live hemp in the field down to discrete particles of approximately 0.25 inch to 0.50 inch size. The chute 24 is preferably mounted on the harvester 20, as is known by those skilled in the art.

The trailer 30 of the present invention preferably cooperates with or is coupled to the forage harvester 20 such that it can be towed behind the forage harvester 20 and can operate in a field. Optionally, as long as it is capable of remaining in close proximity to forage harvester 20, the trailer 30 can be pulled by another vehicle, or it can be an independently self-propelled vehicle capable of transporting equipment. Trailer 30 can be a single trailer or it can be a series of trailers such as trailer 30a and trailer 30b as shown in FIG. 1A. Additionally, while trailers 30, 30a, and 30b are preferably mobile or movable, they can be temporarily or permanently immovable fixtures positioned at, on, or near field 12. Additionally, while not shown in the Figures, it is anticipated that if mobile components of system 10 cannot be transported about field 12 for any reason, an additional third trailer can be used to collect plant material 500 and carry it to the mobile extraction assembly 40a of system 10.

Positioned on trailer 30 are several components and subassemblies of mobile extraction assembly 40a. Optionally, a feed hopper 32 is positioned on trailer 30 as part of or in addition to mobile extraction assembly 40a to cooperate with and receive plant material from the chute 24 of the forest harvester 20 or from a pre-treatment assembly 480 of the mobile extraction assembly 40a. Alternatively and preferably, mobile components 40a are positioned on first and second trailers 30a and 30b such that the pre-treatment assembly 480 cooperates with and receives plant material from the chute 24 of the forest harvester 20 and an inlet hopper 400 cooperates with and receives pre-treated plant material from pre-treatment assembly 480, as shown in FIG. 1A. The mobile extraction assembly 40a preferably separates crude oil from the plant material to produce a crude oil output. The pre-treatment assembly 480 of the mobile extraction assembly 40a also preferably includes at least one solvent tank 422 capable of holding a solvent 522 or use by the mobile extraction assembly 40a. Multiple solvent tanks may be used if needed.

The additional field-based extraction assembly 40b of the mobile and field-based extraction system 40 are removably or permanently located in the field 12 being harvested. Preferably the field-based extraction assembly is positioned near a power source (not shown). Optionally, the field-based extraction assembly 40b can be also carried by trailer 30 or another vehicle so that they can also be relocated, or the field-based extraction assembly 40b can be mobile components that operate alongside the mobile extraction assembly 40a when using a mobile power source (not shown). The field-based extraction assembly 40b includes a second extraction assembly that receives the crude oil output from the mobile extraction assembly 40a and removes and separates solvent and essential oil from the crude oil.

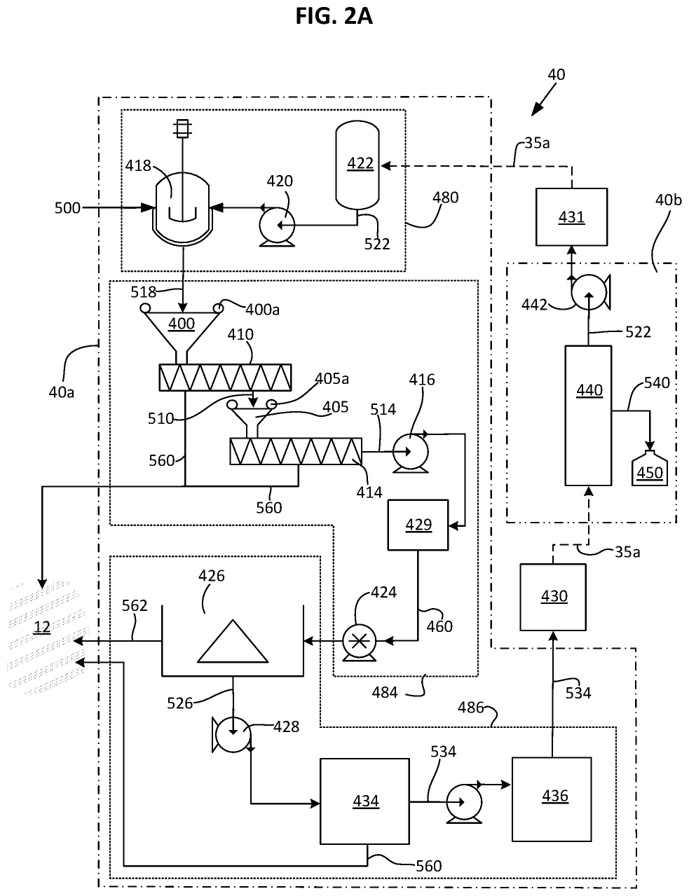

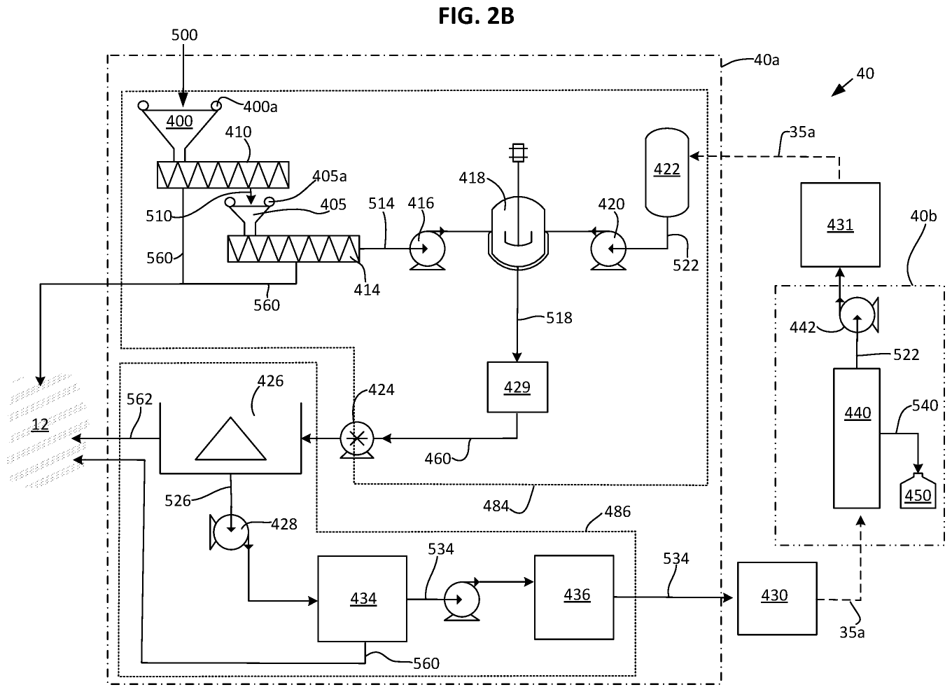

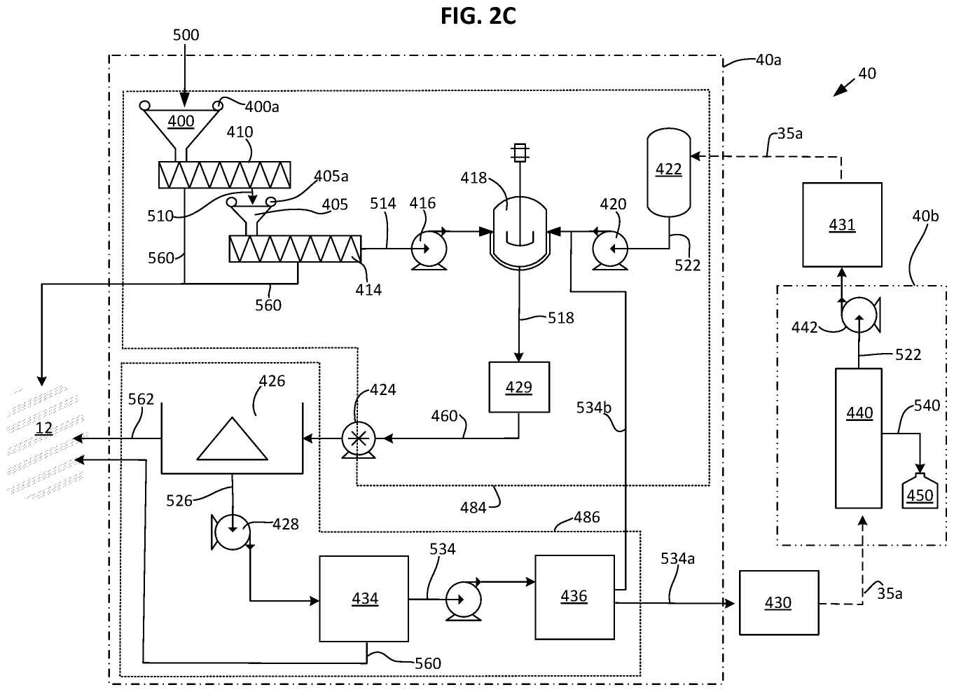

Transporter 35 can be any structure, which includes slides, conveyers, carts, and vehicles, that is capable of moving large objects and materials such as containers of crude oil, solvent, and essential oil between the mobile extraction assembly 40a and the field-based extraction assembly 40b. Preferably, transporter 35 is a self-propelled transport vehicle such as a forklift, as illustrated in FIGS. 1A and 1B, that is capable of moving containers 430 and 431 as reflected on FIGS. 2A-2C by dashed lines labeled 35a.

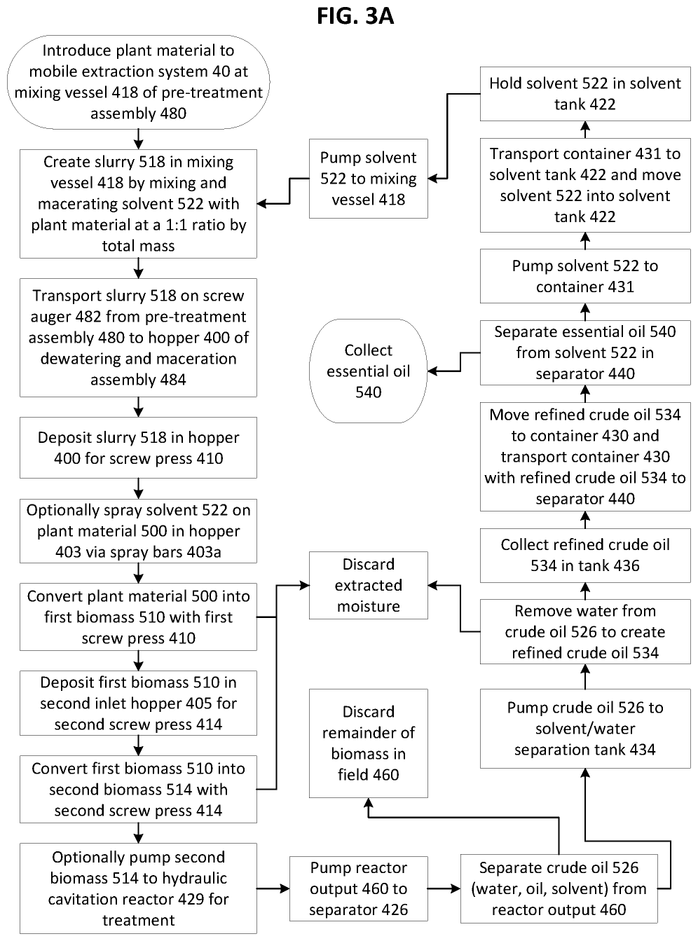

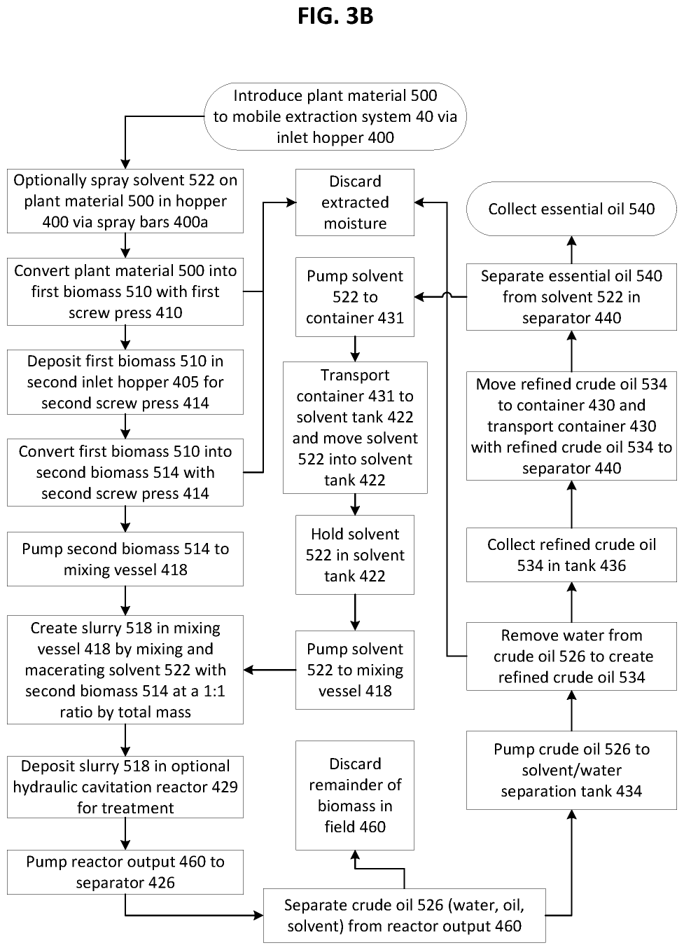

Additional aspects of the mobile and field-based extraction system 40 are illustrated in FIGS. 1A-2C, and its use is outlined in FIGS. 3A and 3B. While the mobile extraction system 40 preferably includes both the mobile extraction assembly 40a and the field-based extraction assembly 40b as described herein, it is anticipated that components of each of assemblies 40a and 40b can be located in either of assemblies 40a and 40b or all of the components can be housed together in alternative embodiments.

As shown and described in FIG. 1A, mobile extraction assembly 40a is preferably positioned on first and second trailers 30a and 30b. Optionally, all of the mobile extraction assembly can be positioned on a single trailer 30 and can include one or more housings in which all of its components can be placed to protect them from environmental damage. Mobile extraction assembly 40a preferably includes three subassemblies: a pre-treatment assembly 480 positioned on first trailer 30a, a dewatering and maceration assembly 484 positioned on second trailer 30b, and a first separation assembly 486 positioned on second trailer 30b, which is shown and described in FIGS. 1A, 2A, and 3A. Where mobile extraction assembly 40a is positioned on multiple trailers, the trailers can be simultaneously towed by harvester 20, towed by separate vehicles, or both or either remain stationary on or near field 12. Additionally, pre-treatment assembly 480 can be permanently positioned on or near field 12 rather than be movable, in which case a third trailer can be used to collect chopped plant material 500 and carry it to pre-treatment assembly 480. While including pre-treatment assembly 480 is preferred, additional embodiments of mobile extraction assembly 40a include the pre-treatment assembly components among the dewatering and maceration assembly 484, as shown in FIGS. 2B-2C and described in FIG. 3B, which would preferably all be positioned on a single trailer 30.

Pre-treatment assembly 480 includes a mixing vessel 418 for receiving plant material 500 from forage harvester 20 via its chute 24, a solvent tank 422 that supplies mixing vessel 418 with solvent 522, and a screw auger conveyer 482 that delivers a slurry 518 output from mixing vessel 418 to an inlet hopper 400 (with or without optional spray bars 400a) positioned among the remaining mobile extraction components such that it cooperates with dewatering and maceration assembly 484, which is shown in FIGS. 1A and 2A. Alternatively, a feed hopper 32 can be positioned on trailer 30 to cooperate with and receive plant material 500 from forest harvester 20 via its chute 24 and deliver plant material to components of the mobile extraction assembly components 40a via a conveyer 32b as shown in FIG. 1B. With respect to both the inlet hopper 400, feed hopper 32, and mixing vessel 418 of pre-treatment assembly 480, they are preferably sized to receive and hold a continuous supply of plant material 500 as forest harvester 20 shears and chops plants grown in the field. Additionally, inlet hopper 400, feed hopper 32, and mixing vessel 418 are further configured to directly or indirectly facilitate transferring plant material 500 or slurry 518 continuously, at intervals, or on demand to a dewatering and maceration assembly 484 of the mobile extraction assembly.

Pre-treatment assembly 480 of mobile extraction assembly 40a also includes solvent tank 422 positioned on trailer 30 or first trailer 30a, optionally in or on a mobile extraction assembly housing. As shown in FIG. 1A, pre-treatment assembly 480 including solvent tank 422 is positioned on first trailer 30a. Solvent tank 422 preferably holds an adequate supply of solvent 522 to support processing of the plant material 500 by mobile extraction assembly 40a. Optionally, solvent tank 422 is in communication with additional components of mobile extraction assembly 40a or field-based extraction assembly 40b to facilitate processing of the plant material 500 as needed. Solvent tank 422 is in communication directly or via a pump with the mixing vessel 418 of pre-treatment assembly 480. Optionally, solvent tank 422 is in communication directly or via pump with dewatering and maceration assembly 484, especially where the components of pre-treatment assembly are included among dewatering and maceration assembly 484 as shown in FIGS. 2B and 2C. Also optionally, solvent tank 422 is in direct or indirect communication with one or more of solvent spray bars 32a, 400a, and 405a.

FIGS. 2A-2C also illustrate the dewatering and maceration assembly 484 of mobile extraction assembly 40a, which includes or is in communication with inlet hopper 400. Inlet hopper 400 preferably is positioned directly above components of the dewatering and maceration assembly 484 of mobile extraction assembly 40a. In particular, in a first and preferred embodiment of dewatering and maceration assembly 484 and as shown in FIG. 2A, inlet hopper 400 is positioned such that it cooperates or communications with mixing vessel 418. More preferably, inlet hopper 400 cooperates and communications with screw auger 482, which delivers slurry 518 from mixing vessel 418 to hopper 400. In a second embodiment of dewatering and maceration assembly 484 and as shown in FIGS. 2B and 2C, inlet hopper 400 is positioned such that it cooperates or communicates with a series of screw presses 410, 414 to allow plant material collected in hopper 400 to be physically moved or transferred to the first screw press 410 of the series of screw presses for further processing. Alternatively, where feed hopper 32 is present, as shown in FIG. 1B, feed conveyor 32b can deliver the plant material 500 from feed hopper 32 (optionally modified with spray bars 32a) to the first screw press 410 of the series of screw presses of the dewatering and maceration assembly 484 or mixing vessel 418 of pre-treatment assembly 480.

Preferably, screw presses 410, 414 are horizontal cone type auger presses or any type of dewatering screw press that optimally use 210,000 to 250,000 lbs.-inch of torque. In the preferred embodiment of mobile extraction assembly 40a and dewatering and maceration assembly 484, the series of screw presses are stacked with additional hoppers 405 (with or without optional spray bars 405a) so that the screw presses are in communication with each other as shown in FIGS. 2A-2C. Alternatively, the screw presses can be connected with conveyers (not shown) to facilitate communication from one screw press to another.

For the second embodiment of mobile extraction assembly 40a and dewatering and maceration assembly 484 and as shown in FIGS. 2B and 2C, after receiving plant material 500 directly from forest harvester 20 via chute 24 and inlet hopper 400 or feed hopper 32, a first screw press 410 presses the plant material 500 into a first biomass 510. For the first and preferred embodiment of the mobile extraction assembly 40a and dewatering and maceration assembly 484 and as shown in FIG. 2A, inlet hopper 400 receives slurry 518 from mixing vessel 418 of pre-treatment assembly 480 via screw auger 482, which is positioned in communication between mixing vessel 418 and inlet hopper 400 (with optional spray bars 400a). Inlet hopper 400 directly communicates with the first screw press 410, which presses the plant material 500 into first biomass 510.

Next, for both the first and second embodiments of mobile extraction assembly 40a and dewatering and maceration assembly 484, through either direct communication with a second screw press 414 via a second inlet hopper 405 (optionally equipped with second spray bars 405a) or indirect communication with the second screw press 414 via a screw conveyer 412, the first biomass 510 output of the first screw press 410 is delivered to screw conveyer 412 for further processing. The second screw press 414 further presses the first biomass 510 to produce a second biomass 514. The second biomass 514 is a press cake with a reduced moisture content compared to the moisture content of the beginning plant material. While the series of screw presses described herein includes only first and second screw presses 410 and 414, it is anticipated that additional screw presses, screw conveyers, and inlet hoppers can be included in the series to produce a press cake with greater moisture reduction. Preferably, the series of screw presses is capable of turning 85% moisture content plant material into 50% moisture content biomass. The moisture 560 removed from the plant material 500 from the series of screw presses will be discarded into the field 12. The extracted moisture 560 preferably is discarded to the field 12 immediately, or it may be collected in a container or other receptacle for further use.

Each of the hoppers 32, 400, and 405 optionally include spray bars 32a, 400a, and 405a, which can be mounted thereon as shown in the Figures. Spray bars hold solvent or any treatment solution that might aid in processing of plant material 500. Preferably, where spray bars are used, the spray bars are configured to deliver solvent or a treatment solution to the contents of the hopper on which or near which the spray bars are positioned. For example, spray bars 32a are preferably positioned along upper edges of feed hopper 32 and are configured to deliver solvent or treatment solution to plant material 500 when it is present in hopper 32. Also preferably, the spray bars are in communication with a source of solvent or pretreatment solution. For example, spray bars 400a could be in communication with a solvent tank 422 that also supplies a solvent 522 to components of mobile processing assembly 40a.

The second embodiment of dewatering and maceration assembly 484 further includes mixing vessel 418, as shown in FIGS. 2B and 2C. The second biomass output 514 is delivered directly or pumped to mixing vessel 418 with an optional first pump 416, all of which are connected and in communication with each other. Mixing vessel 418 also cooperates or communicates with solvent feed tank 422 such that solvent 522 can be transferred from solvent feed tank 422 to mixing vessel 418. Optionally a second pump 420 facilitates the connection between solvent feed tank 422 and mixing vessel 418 so that pump 420, solvent feed tank 422, and mixing vessel 418 are connected and in communication with each other. Preferably first and second pumps 416 and 420 are centrifugal pumps.

Solvent 522 is preferably is an organic or a non-polar solvent. For example, solvent 522 may be n-butanol. Alternatively, solvent 522 may be a hydrogen-infused aqueous solution and optionally one with a pH of 12. Solvent 522 also can be derived from common food products that mimic the properties of non-polar solvent, which preferably can be reused several times. Preferably, solvent 522 is mixed at an approximate 1:1 ratio by total mass to second biomass output 514. Solvent 522 can be mixed with second biomass 514 at additional ratios, however, including at ratios of 1:5 by total mass. Biomass 514 macerates in solvent 522 in mixing vessel 418 for 10 to 20 minutes and becomes a slurry 518.

For the second embodiment of mobile extraction assembly 40a and dewatering and maceration assembly 484, slurry 518 can be directly transferred or optionally pumped from mixing vessel 418 to first separation assembly 486 with a third pump 424, which are all connected and in fluid communication, or it can be subjected to sonication extraction (not shown) or ultrasound assisted extraction (not shown) to reduce use of solvent 522. Additionally and as shown, however, mixing vessel 418 can be in communication with a hydrodynamic cavitation reactor 429 so that slurry 518 can undergo further treatment before being pumped as reactor output 460 to first separation assembly 486. Third pump 424 is preferably a progressive cavity pump.

For the first and preferred embodiment of mobile extraction assembly 40a, which is shown and described in FIGS. 1A, 2A, and 3A, pre-treatment assembly 480, which includes mixing vessel 418, optional pump 420, and solvent feed tank 422, is positioned to immediately cooperate with plant material 500 before it is delivered via screw auger 482 to the series of screw presses 410, 412. Specifically, mixing vessel 418 directly receives plant material 500 from harvester 20. Accordingly, before plant material 500 is delivered to the series of screw presses, it macerates with solvent 522 in mixing vessel 418. Solvent 522 is mixed at an approximate 1:1 ratio by total mass to plant material 500, which can be adjusted based on genetics and density of plant material 500 if needed and as described above. For example, 5 wet pounds of plant material 500 can be mixed with 1 pound solvent 522 or 1 dry pound of plant material 500 can be mixed with 1 pound solvent 522. The biomass macerates in the solvent for 10 to 20 minutes and becomes a slurry 518 or mixture of plant material, solvent, and water before reaching screw presses 410, 412 via intermediary inlet hopper 403.

For the first embodiment of mobile extraction assembly 40a and as with the second embodiment of mobile extraction assembly 40a, the series of screw presses are optionally in communication with first pump 416. For the second embodiment, however, the output 514 of second screw press 414 is transferred next to first separation assembly 486 directly or optionally via a third pump 424, which are all connected and in fluid communication. Alternatively, it can be subjected to sonication extraction (not shown) or ultrasound assisted extraction (not shown) to reduce use of solvent 522. Additionally and as shown, however, second screw press 414 can be in communication a hydrodynamic cavitation reactor 429 directly or via an optional first pump 416 so that second biomass 514 can undergo further treatment before being pumped as reactor output 460 to first separation assembly 486 with third pump 424.

First separation assembly 486 is in communication with dewatering and maceration assembly 484 directly or via and optional third pump 424 to receive slurry 518, biomass output 514, or reactor output 460 and separate out a first crude oil 526. First separation assembly 486 includes a first separator 426 that is in communication with third pump 424 or dewatering and maceration assembly 484. First separator 426 is preferably a decanter centrifuge that is optionally modified with a floater scalping disk for enhanced performance. Alternatively, it can be an ambient filtration skid. First separator 426 separates a first crude oil (water, crude oil, fat, waxes, terpenes, and solvent) 526 from the biomass in slurry 518. The first crude oil 526 communicates directly with or is optionally pumped from first separator 426 with an optional fourth pump 428 to a separation tank 434, and the remaining 45 microns +/− biomass 562 is discarded in the field 12. First separator 426, optional fourth pump 428, and separation tank 434 are all in communication to facilitate the transfer or movement of first crude oil 526. Fourth pump 428 preferably is a transfer or centrifugal pump. Crude oil may be extracted about 25-35 minutes after the plant material was initially chopped, and optimally, the crude oil is extracted only 25 minutes after the plant material 500 was initially chopped.

The solvent/water separation tank 434 of the first separation assembly 486 removes the remaining water or moisture 560 from the first crude oil 526, leaving only a combination of substantially oil and solvent referred to herein as second or refined crude oil 534. The water or moisture 560 is collected in a container (not shown) in communication with tank 434, or it can be immediately discarded in field 12. The refined crude oil 534 is preferably collected in a fixedly or removably attached collection tank 436. Separation tank 434 and tank 436 can be in immediate or direct communication with each other, or they can be indirectly connected through an additional pump (not labeled), as shown in the Figures. Tank 436 preferably holds the refined crude oil 534 until it is ready for further processing, at which time it can be transferred to a movable intermediate bulk container 430 or introduced to the field-based extraction assembly 40b through direct or indirect connection or communication. Alternatively, refined crude oil 534 can be divided such that a first part 534a of refined crude oil 534 will be transferred to removable bulk container 430 or field-based extraction assembly 40b and a second part 534b of refined crude oil 534 will recirculate back to mix tank 418 to increase the saturation of the oil in the solvent and depending on whether there was full saturation on the first pass. FIG. 2C illustrates and optional configuration of the second embodiment of the mobile extraction assembly where first portion of refined crude oil 534a is directed to container 430 and second portion of refined crude oil 534b is directed back to mix tank 418. To facilitate transferring a portion of its contents to mix tank 418, tank 436 is in communication with mix tank 418 directly or optionally indirectly using an additional pump (not shown).

Field-based extraction assembly 40b includes a second separator 440, which receives the refined crude oil 534 or the first portion of refined crude oil 534a. Refined crude oil 534 or first portion of refined crude oil 534a can either be pumped to second separator 440 with a sixth pump (not shown) where all components of the extraction system are mobile or, more preferably, transported in movable container 430 to the second separator 440 and field-based components 40b with transporter 35. Preferably, second separator 440 is an oil and solvent separator 440 and more preferably is a wiped film evaporator. Second separator 440 separates essential oil 540 from solvent 522 and is in communication with an essential oil container 450 for receiving and collecting essential oil 540 for further processing. Second separator 440 also is in communication with solvent feed tank 422, optionally through an intermediary pump, or with a reclaimed solvent container or tote 431, also optionally through an intermediary pump. Where second separator 440 is in communication with solvent feed tank 422 such as when field-based extraction assembly 40b is also mobile, the reclaimed solvent 522 can be immediately reused by the mobile extraction assembly 40a. Alternatively and as shown in the Figures, movable container 431 is configured such that it can receive and collect reclaimed solvent 522 and be transported back to solvent feed tank 422 with transporter 35 or another transporter. The time it takes to remove the essential oil 540 from the crude oil is about 15 to 25 minutes. Optimally, it takes only 15 minutes to separate the essential oil 540. The total time to process the cut plant material and recover essential oil 540 is therefore about 40 to 60 minutes.

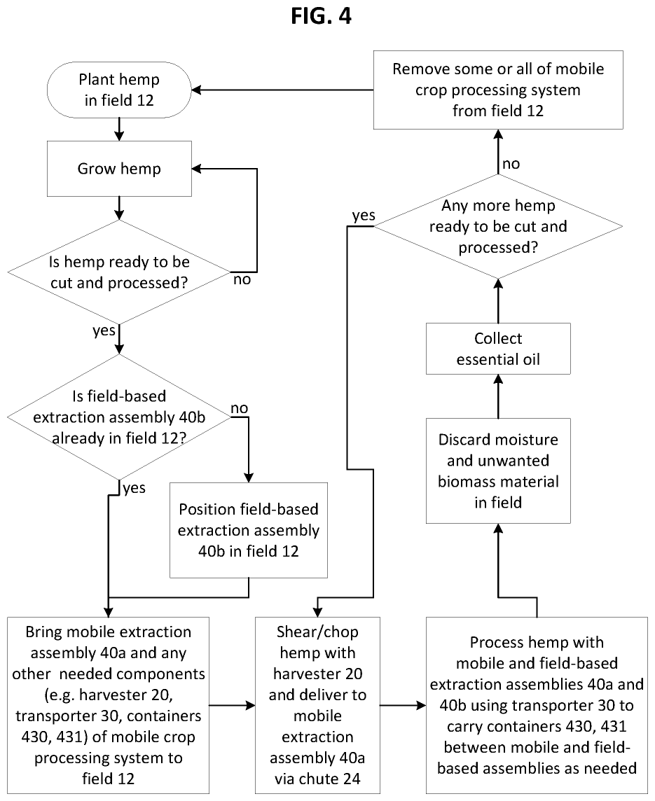

The system described herein can be used in the field to expedite the moisture removal from plant material such as industrial hemp and then to extract the essential oils, as illustrated in FIG. 4. Specifically, a method of farming industrial hemp to produce essential oils using the system described herein requires first planting industrial hemp seeds or seedlings in approximately 120 acre increments. Optimally, after about 90 days' time, the hemp is ready to be cut and processed. To do so, a farmer uses the forage harvester with chopper described herein to chop the hemp into small pieces and deliver to the mobile extraction assembly 40a via an inlet hopper, a feed hopper, or mixing vessel on the trailer. Initially, the hopper or mixing vessel is filled part way and then a continuous operation is started. As the feed hopper or mixing vessel fills, it delivers the chopped hemp to the mobile extraction assembly 40a of the mobile crop processing system, which cooperates with the field-based extraction assembly 40b to remove moisture and unwanted biomass and extract and essential oil with a series of screw presses, extractors, and separators as described above. Additionally, as needed, transporter 35 delivers containers of solvent 522 and crude oil 534 between the mobile components 40a and field-based components 40b of mobile and field-based extraction system 40. During processing of the hemp, unwanted moisture and biomass is discarded in the field or collected if desired, solvent is recycled, and essential oil is collected. The mature industrial hemp can be processed using the system described herein at a rate of 4000-8000 lbs./hour of plant mass. Accordingly, it would take approximately 60-120 hours to process 120 acres of plant material in the field. During processing, for an initial plant moisture content of 75%-80% and estimated 27,000 lbs. of oil in 120 acres of plant material, the anticipated essential oil recovered per hour is approximately 220 lbs. Over an entire year of repeatedly planting and harvesting the plant material, it is anticipated that the farmer should recover 20,647 lbs. of essential oil at a yield rate of 76% for approximately 120 acres of farmed land.

There are several possible advantages to using the farming method and mobile crop processing system of the present invention. For example, farmers may find processing is safer as solvent extraction methods with a lower flammability class can be used in an outside environment thereby limiting hazards to workers. Additionally, the smaller footprint of the mobile crop processing system allows for processing about 1 acre of industrial hemp per hour, which reduces harvest and processing costs compared to traditional drying and processing systems. Processing in the field also may facilitate quick capture of the natural profiles of the hemp plant (tricomes, terpenes, CBDA). Finally, the ability to move the mobile crop processing system between fields allows for multiple operations to be harvested in a single season.

While in the foregoing, embodiments of the present invention have been set forth in considerable detail for the purposes of making a complete disclosure of the invention, it may be apparent to those of skill in the art that numerous changes may be made in such detail without departing from the spirit and principles of the invention.

We claim:

1) A mobile crop processing system comprising:
a) a self-propelled harvester configured to shear and chop plant material located in a field;
b) a mobile extraction assembly capable of movement about the field and positioned adjacent the harvester to receive the plant material from the harvester, wherein the mobile extraction assembly causes a crude oil output and comprises:
i) a dewatering and maceration assembly; and
ii) a first separation assembly in communication with the dewatering and maceration assembly;
c) a movable first container in communication with the first separation assembly of the mobile extraction assembly, wherein the container is configured to receive and collect the crude oil output of the mobile extraction assembly; and
d) a field-based extraction assembly positioned at the field, wherein the field-based extraction assembly causes an essential oil output and comprises a second separation assembly in communication with the first movable container to receive the crude oil output of the mobile extraction assembly.

2) The mobile crop processing system of claim 1, wherein the mobile extraction assembly further comprises a pre-treatment assembly comprising:
a) a mixing vessel positioned to receive plant material from the harvester;
b) a solvent tank in communication with the mixing vessel; and
c) a screw auger in communication with the mixing vessel and the dewatering and maceration assembly, wherein the screw auger is positioned to receive and carry a slurry output from the mixing vessel to the dewatering and maceration assembly.

3) The mobile crop processing system of claim 2 further comprising a transporter positioned on the field, wherein the transporter is capable of carrying the first movable container between the mobile extraction assembly and field-based extraction assembly.

4) The mobile crop processing system of claim 2 further comprising:
a) a second movable container in communication with the second separation assembly of the field-based extraction assembly, wherein the second movable container is configured to receive and collect a solvent output from the field-based extraction assembly; and
b) a transporter positioned on the field, wherein the transporter carries the first and second movable containers between the mobile extraction assembly and field-based extraction assembly.

5) The mobile crop processing system of claim 1 wherein the dewatering and maceration assembly comprises:
a) a hopper positioned to receive the plant material from the harvester;
b) a series of screw presses in communication with the hopper; and
c) a mixing vessel in communication with the series of screw presses, a solvent tank, and the first separation assembly.

6) The mobile crop processing system of claim 2 wherein the dewatering and maceration assembly comprises:
a) a hopper positioned to receive a slurry from the pre-treatment assembly; and
b) a series of screw presses positioned between and in communication with the hopper and the first separation assembly.

7) The mobile crop processing system of claim 6 wherein the dewatering and maceration assembly further comprises a hydrodynamic cavitation reactor positioned between and in communication with the mixing vessel and the first separation assembly.

8) The mobile crop processing system of claim 2 wherein the first separation assembly comprises:
a) a separator in communication with the series of screw presses of the dewatering and maceration assembly;
b) a separation tank in communication with the separator; and
c) a collection tank in communication with the separation tank and the movable first container.

9) The mobile crop processing system of claim 8 wherein the separator comprises a decanter centrifuge.

10) The mobile crop processing system of claim 8 wherein the separator comprises an ambient filtration skid.

11) The mobile crop processing system of claim 6 wherein the second separation assembly comprises a wiped film evaporator.

12) The mobile crop processing system of claim 3 wherein the transporter is a self-propelled transport vehicle.

13) The mobile crop processing system of claim 1 further comprising a trailer coupled to the harvester wherein the mobile extraction assembly is position on the trailer.

14) The mobile crop processing system of claim 2 further comprising first and second trailers coupled to the harvester, wherein the pre-treatment assembly of the mobile extraction assembly is positioned on the first trailer and the dewatering and maceration assembly and first separation assembly of the mobile extraction assembly are positioned on the second trailer.

15) A mobile crop processing system comprising:
a) a self-propelled harvester configured to shear and chop plant material located in a field;
b) a mobile extraction assembly capable of movement about the field and positioned adjacent the harvester to receive the plant material from the harvester, wherein the mobile extraction assembly causes a crude oil output and comprises:
i) a pre-treatment assembly comprising a mixing vessel positioned to receive plant material from the harvester, a solvent tank in communication with the mixing vessel, and a screw auger in communication with the mixing vessel and positioned to receive a slurry output from the mixing vessel;
ii) a dewatering and maceration assembly comprising a hopper positioned to receive the slurry from the screw auger and a series of screw presses positioned between and in communication with the hopper and a first separation assembly; and
iii) the first separation assembly comprising a separator in communication with the series of screw presses, a separation tank in communication with the separator, and collection tank in communication with the separation tank and positioned to receive a crude oil output from the separation tank;
c) a movable first container in communication with the collection tank of first separation assembly, wherein the first container is configured to receive and collect the crude oil output of the first separation assembly; and
d) a field-based extraction assembly positioned at the field, wherein the field-based extraction assembly causes an essential oil output and comprises a wiped film evaporator in communication with the first movable container to receive the crude oil output of the mobile extraction assembly, a movable second container in communication with the wiped film evaporator and positioned to receive and collect reclaimed solvent output from the wiped film evaporator, and a third container in communication with the wiped film evaporator and positioned to receive and collect essential oil output from the wiped film evaporator; and
e) a transporter positioned on the field, wherein the transporter is capable of carrying the first and second movable containers between the mobile extraction assembly and field-based extraction assembly.

16) The mobile crop processing system of claim 15 further comprising first and second trailers, wherein the first trailer is coupled to the harvester, the second trailer is coupled to the first trailer, the pre-treatment assembly of the mobile extraction assembly is positioned on the first trailer, and the dewatering and maceration assembly and first separation assembly of the mobile extraction assembly are positioned on the second trailer.

17) A method of harvesting and processing plant material in a field to produce essential oil, the method comprising:
a) introducing a mobile crop processing system to the field, wherein the mobile crop processing system comprises:
i) a self-propelled harvester configured to shear and chop the plant material located in the field;
ii) a first trailer coupled to the harvester;
iii) a mobile extraction assembly positioned on the first trailer;
iv) a field-based extraction assembly positioned on the field;
v) a transporter vehicle located on the field and capable of transporting containers between the mobile extraction assembly and field-based extraction assembly;
b) shearing and chopping the plant material with the harvester;
c) delivering the chopped plant material to the mobile extraction assembly;
d) activating the mobile extraction assembly to produce crude oil from the plant material and a solvent;
e) collecting the crude oil in a first container;
f) using the transporter, transporting the first container from the mobile extraction assembly to the field-based extraction assembly; and
g) activating the field-based extraction assembly to produce essential oil and reclaimed solvent from the crude oil.

18) The method of claim 17 further comprising:
a) collecting the reclaimed solvent in a second container;
b) using the transporter, transporting the second container from the field-based extraction assembly to the mobile extraction assembly; and
c) transferring the reclaimed solvent from the second container to a solvent feed tank of the mobile extraction assembly.

19) The method of claim 17 further comprising a second trailer coupled to the harvester, wherein the mobile extraction assembly comprises a pre-treatment assembly positioned on the first trailer that receives the chopped plant material, a dewatering and maceration assembly positioned in communication with the pre-treatment assembly and positioned on the second trailer, and a first separation assembly in communication with the dewatering and maceration assembly that outputs the crude oil and is positioned on the second trailer.

20) The method of claim 17 further comprising discarding excess moisture and biomass in the field while operating the mobile extraction assembly.
